(19) United States
(12) Patent Application Publication
(10) Pub. No.: US 2022/0325356 A1
(43) Pub. Date: Oct. 13, 2022

(54) METHOD FOR ALTERING THERAPY OF ADVANCED NON-SMALL CELL LUNG CANCER PATIENTS BASED ON ANALYSIS OF ctDNA

(71) Applicant: Inivata Ltd., Cambridge (GB)

(72) Inventor: Clive Morris, Cambridge (GB)

(21) Appl. No.: 17/634,217

(22) PCT Filed: Jul. 31, 2020
(86) PCT No.: PCT/IB2020/057236

§ 371 Date: Feb. 9, 2022

Related U.S. Application Data
(60) Provisional application No. 62/886,215, filed on Aug. 13, 2019.

(51) Int. Cl. C12Q 1/6886; A61K 38/17

(52) U.S. Cl. CPC C12Q 1/6886; A61K 38/1774; C12Q 2600/106

(57) ABSTRACT

Provided herein is a method of treating non-small cell lung cancer. In some embodiments, the method may comprise monitoring the amount of ctDNA in a patient that is undergoing treatment by pembrolizumab monotherapy for non-small cell lung cancer, identifying the patient as having increasing ctDNA, and administering an effective amount of pembrolizumab and platinum-based doublet chemotherapy to the identified patient.

### BACKGROUND

Lung cancer is a leading cause of cancer-related death, both in the United States and worldwide. At least 80 percent of lung cancers are classified as non-small cell lung cancer (NSCLC) and, as such, there is a great need for treatment strategies for NSCLC.

NSCLC patients that have a “druggable” genetic alteration (e.g., an alteration in epidermal growth factor receptor (EGFR) or anaplastic lymphoma kinase (ALK)) can be treated with a targeted therapy. However, the majority of NSCLC patients lack such genetic alterations and, as such, targeted therapies are not effective for most NSCLC patients.

Monotherapy using the immune checkpoint inhibitor pembrolizumab (a humanized anti-PD-1 antibody) has become the frontline treatment for patients with non-targetable tumors that are associated a high level of PD-L1 expression. Approximately 30% of NSCNC patients have such tumors. However, many patients, e.g., those that have a rapidly progressive disease or a high tumor burden, do not completely respond to pembrolizumab monotherapy and do not have a good clinical outcome.

Better methods for identifying patients that do not completely respond to pembrolizumab monotherapy and more aggressive ways for treating such patients are therefore needed.

### SUMMARY

Provided herein is a method of treating non-small cell lung cancer. In some embodiments, the method may comprise monitoring the amount of ctDNA in a patient that is undergoing treatment by pembrolizumab monotherapy for non-small cell lung cancer, identifying the patient as having increasing ctDNA, and administering an effective amount of pembrolizumab and platinum-based doublet chemotherapy to the identified patient. The amount of ctDNA in the patient may be measured in a variety of different ways. In some embodiments, the monitoring may comprise measuring the variant allele fraction of at least one variant sequence in a sample of cfDNA from the patient, before and after treatment the first administration of pembrolizumab to the patient. In other embodiments, the total amount of mutant DNA (as measured by estimating the number of mutant copies of the patient's genome) per volume of plasma can be measured, before and after treatment the first administration of pembrolizumab to the patient. If the variant allele fraction of the at least one variant sequence or the total amount of mutant DNA increases after the first administration of pembrolizumab, then the patient can be treated more aggressively using a combination of pembrolizumab and platinum-based doublet chemotherapy. If the variant allele fraction or the total amount of mutant DNA decreases or stays approximately the same after treatment by pembrolizumab monotherapy, then the patient can stay on pembrolizumab monotherapy.

Because the effectiveness of pembrolizumab monotherapy can be readily determined by analyzing ctDNA, the effectiveness of the treatment can be determined relatively quickly and, if the patient is not responding, then he or she can be immediately treated more aggressively with pembrolizumab and platinum-based doublet chemotherapy.

These and other features of the present teachings are described herein.

### Definitions

Before describing exemplary embodiments in greater detail, the following definitions are set forth to illustrate and define the meaning and scope of the terms used in the description.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The terms “next-generation sequencing” or “high-throughput sequencing”, as used herein, refer to the so-called parallelized sequencing-by-synthesis or sequencing-by-ligation platforms currently employed by Illumina, Life Technologies, and Roche, etc. Next-generation sequencing methods may also include nanopore sequencing methods such as that commercialized by Oxford Nanopore Technologies, electronic-detection based methods such as Ion Torrent technology commercialized by Life Technologies, or single-molecule fluorescence-based methods such as that commercialized by Pacific Biosciences.

The term “sequencing at least part of the coding sequences” refers sequencing at least 20% of, at least 40% of, at least 60% of, at least 80% of, or at least 90% of (e.g., all of), of the coding sequences.

The term “reference sequence”, as used herein, refers to a known nucleotide sequence, e.g. a chromosomal region whose sequence is deposited at NCBI's Genbank database or other databases, for example. A reference sequence can be a wild type sequence.

As used herein, the terms “cell-free DNA from the bloodstream” and “circulating cell-free DNA” refers to DNA that is circulating in the peripheral blood of a patient. The DNA molecules in cell-free DNA have a median size that is below 1 kb (e.g., in the range of 50 bp to 500 bp, 80 bp to 400 bp, or 100-1,000 bp), although fragments having a median size outside of this range may be present. Cell-free DNA may contain circulating tumor DNA (ctDNA), i.e., tumor DNA circulating freely in the blood of a cancer patient. cfDNA can be obtained by centrifuging whole blood to remove all cells, and then isolating the DNA from the remaining plasma or serum. Such methods are well known (see, e.g., Lo et al, Am J Hum Genet 1998; 62:768-75). Circulating cell-free DNA can be double-stranded or single-stranded.

As used herein, the term “circulating tumor DNA” (or “ctDNA”) is tumor-derived DNA that is circulating in the peripheral blood of a patient. ctDNA is of tumor origin and originates directly from the tumor or from circulating tumor cells (CTCs), which are viable, intact tumor cells that shed from primary tumors and enter the bloodstream or lymphatic system then die. The precise mechanism of ctDNA release is unclear, although it is postulated involve apoptosis and necrosis from dying cells, or active release from viable tumor cells. ctDNA can be highly fragmented and in some cases can have a mean fragment size about 100-250 bp, e.g., 150 to 200 bp long. The amount of ctDNA in a sample of circulating cell-free DNA isolated from a cancer patient varies greatly: typical samples contain less than 10% ctDNA, although many samples have less than 1% ctDNA and some samples have over 10% ctDNA. Molecules of ctDNA can be often be identified because it contains tumorigenic mutations.

As used herein, the terms “treat”, “treatment” and “treating” or the like herein refers to administering a compound or pharmaceutical composition as provided herein for therapeutic purposes. A treatment involves administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

As used herein, the term “therapeutically effective amount” refers to the amount of a compound that, when administered to a patient having a disease, is sufficient to effect such treatment for the disease. The “therapeutically effective amount” will vary depending on the compound, the disease and its severity and the age, weight, etc., of the subject to be treated.

As used herein, the term “actionable sequence variation” is a sequence variation for which there is a therapy that specifically targets the activity of the protein having the variation. In many embodiments an actionable sequence variation causes an increase in an activity of the protein, thereby resulting in cells containing the variation to grow, divide and/or metastasize without check and in combination with other variations, such as in tumour suppressor genes, leading to cancer.

As used herein, the term “therapy that is targeted to an actionable sequence variation” is a therapy that targets the activity of the protein having the sequence variation. Therapy that is targeted to an actionable sequence variation often inhibits an activity of the mutated protein. Examples of actionable sequence variations for non-small cell lung cancer and some other cancers, as well as therapies that target those actionable variations, are known. For non-small cell lung cancer, actionable variants are most commonly found in EGFR, ALK, ROS1, and BRAF, where the actionable variations in EGFR and BRAF are activating mutations, the actionable variations in ALK and ROS1 are gene fusions.

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, the some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patients and publications, including all sequences disclosed within such patients and publications, referred to herein are expressly incorporated by reference.

As noted above, a method of treating non-small cell lung cancer is provided. In some embodiments, the method may comprise monitoring the amount of ctDNA in a patient that is undergoing treatment by pembrolizumab monotherapy (i.e., being treated with pembrolizumab only, without additional treatment with a chemotherapic agent or a kinase inhibitor) for non-small cell lung cancer, identifying the patient as having increasing ctDNA, and administering an effective amount of pembrolizumab and platinum-based doublet chemotherapy to the identified patient. In many embodiments, the non-small cell lung cancer is PDL1 positive and is not associated with any actionable sequence variation. For example, the cancer may be PD-L1 positive and may have wild type EGFR, ALK, ROS1, and BRAF genes.

In some embodiments, the amount of ctDNA may be monitored by measuring the variant allele fraction of at least one sequence variant in a sample of cfDNA from the patient, before and after treatment by pembrolizumab. For example, the amount of ctDNA may be monitored by i. measuring the variant allele fraction of at least one variant sequence within one week of the first administration of pembrolizumab to the patient (e.g., within one week before treatment); ii. independently measuring the variant allele fraction of the variant sequence two to four weeks after the first administration of pembrolizumab to the patient; and iii. comparing the measurements. In other embodiments, the total amount of mutant DNA per a unit volume of plasma can be measured. For example, the number of mutant copies of the patient's genome in a volume (e.g., 1 ml) of plasma can be determined within one week of the first administration of pembrolizumab to the patient (e.g., within one week before treatment); ii. independently measuring the number of mutant copies of the patient's genome in the same volume (e.g., 1 ml) of plasma two to four weeks after the first administration of pembrolizumab to the patient; and iii. comparing the measurements. Comparing the measurements allows one to determine whether the ctDNA is decreasing (which indicates that the pembrolizumab monotherapy is working) staying the same, or increasing (which indicates that the pembrolizumab monotherapy is not working as effectively as needed and the patient should be treated with platinum-based doublet chemotherapy in addition to pembrolizumab). ctDNA molecules can be identified in cfDNA because they contain sequence variations (e.g., a substitution or in-del of one or more nucleotides relative the a reference, e.g., wild-type, sequence) and typically have an allele frequency of less than 10%, often less than 5% or less than 1%. As such, the amount of ctDNA in a sample of cfDNA can be measured by comparing the number of molecules in the cfDNA that have a sequence variation to the the number of molecules in the cfDNA that do not have the sequence variation to calculate the “variant allele fraction”, where the variant allele fraction (which is often expressed as a percentage) indicates the the number of molecules of cfDNA that contain a sequence variation relative to the number molecules of cfDNA that do not contain the sequence variation.

In some embodiments, the sequence variation may be in a cancer-related gene, e.g., KRAS or TP53. However, because many cancers have a high mutational burden (see, e.g., Meléndez et al Transl Lung Cancer Res. 2018 7: 661-667), the sequence variations analyzed in the present method can be in virtually any gene and can also be non coding. The variant allele fraction of a sequence variant can be calculated in a variety of different ways.

For example, in some embodiments, the method may done by sequencing. Such sequencing methods involve counting molecular barcodes or counting the number of sequences reads correspond to the wild type and variant sequences. In some embodiments, the method may involve shotgun sequencing an unenriched/unamplified sample or sequencing the entire exome. In other embodiments, the method does not involve shotgun sequencing an unenriched/unamplified sample or sequencing the entire exome. Rather, the sequencing may be done as part of a larger sequencing effort that targets at least part of the coding sequences for up to 500, e.g., up to 200 or up to 100 or up to 50 genes, focusing on the coding sequences of cancer genes (e.g., AKT1, ALK, BRAF, CCND1, CDKN2A, CTNNB1, EGFR, ERBB2, ESR1, FGFR1, FGFR2, FGFR3, GATA3, GNA11, GNAQ, GNAS, HRAS, IDH1, IDH2, KIT, KRAS, MAP2K1, MET, MYC, NFE2L2, NRAS, NTRK1, NTRK3, PDGFRA, PIK3CA, PPP2R1A, PTEN, ROS1, STK11, TP53 and/or U2AF1) as well as the coding sequences of other genes or mutations which are associated with non-small cell lung cancer. In other embodiments, the sequence variants may be identified on a patient-by-patient basis. In these embodiments, the analysis may be done in two steps. In the first step, the patient's tumour or cfDNA is sequenced and one or more tumor-derived sequence variations are identified. In the second step, the identified sequence variations are analyzed. In these embodiments, the analysis may be targeted to a relatively small number of coding or non coding sequences (e.g., 5-500) that contain mutations. Methods for sequencing target sequences in cfDNA are known and, in some embodiments, the method may comprise enriching for or amplifying target sequences by PCR prior to sequencing (see, e.g., Forshew et al, Sci. Transl. Med. 2012 4:136ra68, Gale et al, PLoS One 2018 13:e0194630 and Weaver et al, Nat. Genet. 2014 46:837-843, among many others). If the sequence variation is a rearrangement such as a gene fusion, then they may be identified using the PCR-based method described in PCT/GB2018/051688, filed on Jun. 18, 2018, and GB1709675.1, filed on Jun. 16, 2017, and quantified using the method described in U.S. provisional application Ser. No. 62/778,537, filed on Dec. 12, 2018, which applications are incorporated by reference herein for disclosure of those methods. In some embodiments, the method may comprise performing an amplification reaction using target-specific primers that flank a sequence variation to produce an amplification product, sequencing the amplification product to produce sequence reads, and then comparing the number of sequence reads that have the sequence variation to the number of sequence reads that do not have the sequence variation. Methods that use molecular barcoding (see, e.g., Casbon et al Nuc. Acids Res. 2011, 22 e81) can also be used. Methods that use hybrid capture to select the region of interest can be used (see, e.g., Murtaza, M., et al. Nature, 497(7447), 108-112).

In some embodiments the method comprises analyzing replicate samples (e.g., 2, 3 or 4 aliquots of the same sample) of the cfDNA at each time point, thereby providing a more accurate way to estimate the variant allele fraction of a sequence variant. In these embodiments, the results obtained for each timepoint may analyzed statistically, thereby providing more confidence in any determination of whether the variant allele fraction of the sequence variant is increasing or decreasing.

A patient that has increasing ctDNA may a patient that has at least a 50% increase (e.g., at least a 60% increase, at least a 70% increase, at least a 80% increase, at least a 90% increase, at least a 100% increase) in ctDNA two to four weeks after the first administration of pembrolizumab to the patient. In some embodiments the amount of ctDNA may be monitored by independently measuring the variant allele fraction of a plurality of sequence variants (e.g., at least 2, at least 5, at least 10 or at least 50 variants) in a sample of cfDNA from the patient, before and after treatment by pembrolizumab.

If a patient is switched onto treatment that includes pembrolizumab and a platinum-based doublet chemotherapy (in which the platinum-based doublet chemotherapy may comprise a platinum-based agent selected from cisplatin (CDDP), carboplatin (CBDCA), and nedaplatin (CDGP)) and one third-generation agent (selected from docetaxel (DTX), paclitaxel (PTX), vinorelbine (VNR), gemcitabine (GEM), irinotecan (CPT-11), pemetrexed (PEM), and tegafur gimeracil oteracil (S1)) then, in these embodiments, pembrolizumab can be administered to the patient as an intravenous infusion (200 mg or 2 mg/kg, up to 200 mg) over 30 minutes, every three weeks. Dosages and timing for platinum-based doublet chemotherapy are also known but vary from combination to combination (see, e.g., Besse et al, Annals of Oncology 2005 16: 997-998 and Sangal et al Lung Cancer Management 2013 2: 5, among many others). As would be apparent, if a patient is switched onto treatment that includes pembrolizumab and a platinum-based doublet chemotherapy, then the treatment schedule for pembrolizumab (i.e., intravenous infusion over 30 minutes, every three weeks) may continue under the same schedule and the platinum-based doublet chemotherapy may be added. In these embodiments the platinum-based doublet chemotherapy may be added. Some platinum-based doublet chemotherapies are administered every three weeks. In these embodiments, the platinum-based doublet chemotherapy may be administered on the same day as the pembrolizumab. In other embodiments, the platinum-based doublet chemotherapy may be administered in between the pembrolizumab administrations.

In some embodiments, the method may further comprise sequencing at least some of the same regions (e.g. amplicons) from white blood cell DNA from the same subject. In these embodiments, the method may involve comparing the genetic variations called using cfDNA to the genetic variations called using the white blood cell DNA. If a variation is identified in the white blood cell DNA, then it may be identified as being a genetic variation with a lower confidence or not all. This embodiment provides a way to identify variations that may be potentially due to clonal hematopoiesis of indeterminate potential (CHIP) (see, generally, Funari et al, Blood 2016 128:3176 and Heuser et al, Dtsch Arztebl Int. 2016 113: 317-322), or may be germ line variants for example.

### EXAMPLES

Aspects of the present teachings can be further understood in light of the following examples, which should not be construed as limiting the scope of the present teachings in any way.

### Example 1

### Sequencing Method

Whole blood is collected into Streck Blood collection tubes (Streck BCT). Upon collection, the Streck BCTs are gently inverted 8-10 times before being shipped immediately. Within 7 days they are centrifuged at 1600×g for 10 minutes at room temperature, plasma is removed, transferred to a new tube and then a 2nd centrifugation step is performed at 20,000×g for 10 minutes to pellet any remaining cellular debris before transferring the plasma to a new tube. Upon completion of processing all cfDNA samples are frozen at −80° C. until ready for analysis.

Cell free DNA is extracted from plasma using the QIAamp Circulating Nucleic Acid kit (Qiagen). Digital PCR is then performed using the BioRad QX200 and an assay targeting a 108 bp region of the ribonuclease P/MRP subunit p30 (RPP30) gene.

Between 2,000 and 16,000 amplifiable copies (as measured by digital PCR) are then used to setup a sequencing library. PCR Primers targeting AKT1, ALK, BRAF, CCND1, CDKN2A, CTNNB1, EGFR, ERBB2, ESR1, FGFR1, FGFR2, FGFR3, GATA3, GNA11, GNAQ, GNAS, HRAS, IDH1, IDH2, KIT, KRAS, MAP2K1, MET, MYC, NFE2L2, NRAS, NTRK1, NTRK3, PDGFRA, PIK3CA, PPP2R1A, PTEN, ROS1, STK11, TP53 and U2AF1 are multiplexed together. These are used to amplify these 36 genes from the cell free DNA. Following PCR, the products are cleaned up using SPRIselect reagent (Beckman Coulter B23319) using the manufacturers protocol. DNA is then eluted in 18 uL. A second round of PCR is performed targeting sequences added during the first PCR. Each primer pair contains a unique barcode combination to enable subsequent sample demultiplexing.

The PCR product was cleaned up once using SPRIselect reagent (Beckman Coulter B23319) using the manufacturers protocol. Indexed samples are pooled into a tube containing 10 uL 10 mM Tris-HCl pH 8. Samples are then size selected for 195-350 bp using a 2% Agarose Dye Free cassette and marker L on the Pippin Prep (Sage Science), following the manufacturer's instructions. Size selected DNA is quantified by Qubit, following the manufacturer's instructions. Quantified libraries are sequenced on the NextSeq500 Illumina platform (300 cycle PE) with 5% PhiX to monitor sequencing performance and data analysis is performed.

Sequencing files are analyzed using the Inivata Somatic Mutation Analysis (ISoMA) pipeline to identify SNVs, CNVs and indels. In the ISoMA pipeline a minimum Phred quality score of 30 for each base is required for inclusion in the analytics. For SNV and indel analysis, a background model is first established using samples from presumed healthy donors for each position/base pair change covered by our panel. The final determination of an SNV call integrates the data across multiple replicates for each sample in comparison with this background within a maximum likelihood framework. The same statistical principle is used for indels using samples from the same analytical batch in order to enable appropriate background calibration. The minimum depth at which any SNV or indel would be called is 1000×.

### Example 2

### Monitoring Response to Therapy

In this example, serial plasma samples are collected beginning at base line prior to initial therapy and then subsequent collections over a period of 2 months. Changes from base line in the allele frequency (AF) of genomic alterations in ctDNA are calculated.

It will also be recognized by those skilled in the art that, while the invention has been described above in terms of preferred embodiments, it is not limited thereto. Various features and aspects of the above described invention may be used individually or jointly. Further, although the invention has been described in the context of its implementation in a particular environment, and for particular applications (e.g. ctDNA analysis) those skilled in the art will recognize that its usefulness is not limited thereto and that the present invention can be beneficially utilized in any number of environments and implementations where it is desirable to examine cfDNA. Accordingly, the claims set forth below should be construed in view of the full breadth and spirit of the invention as disclosed herein.

1. A method of treating non-small cell lung cancer, comprising:
(a) monitoring the amount of ctDNA in a patient that is undergoing treatment by pembrolizumab monotherapy for non-small cell lung cancer;
(b) identifying the patient as having increasing ctDNA; and
(c) administering an effective amount of pembrolizumab and platinum-based doublet chemotherapy to the identified patient.

2. The method of claim 1, wherein the platinum-based doublet chemotherapy comprises a platinum-based agent selected from cisplatin (CDDP), carboplatin (CBDCA), and nedaplatin (CDGP) and one third-generation agent selected from docetaxel (DTX), paclitaxel (PTX), vinorelbine (VNR), gemcitabine (GEM), irinotecan (CPT-11), pemetrexed (PEM), and tegafur gimeracil oteracil (S1).

3. The method of claim 1, wherein step (a) comprises measuring the variant allele fraction of at least one sequence variant in a sample of cfDNA from the patient, before and after the first administration of pembrolizumab to the patient.

4. The method of claim 1, wherein step (a) comprises independently measuring the variant allele fraction of a plurality of sequence variants in a sample of cfDNA from the patient, before and after the first administration of pembrolizumab to the patient.

5. The method of claim 1, wherein step (a) comprises:
(i) measuring the variant allele fraction of at least one sequence variant within one week of the first administration of pembrolizumab to the patient;
(ii) independently measuring the variant allele fraction of the sequence variant two to four weeks after the first administration of pembrolizumab to the patient; and
(iii) comparing the measurements obtained in steps (i) and (ii).

6. The method of claim 1, wherein the method comprises analyzing replicate samples of the cfDNA at each time point.

7. The method of claim 1, wherein the cancer is PD-L1 positive and/or is not associated with any actionable mutations.

8. The method of claim 1, wherein a patient that has increasing ctDNA is a patient that has at least a 50% increase in ctDNA.

9. The method of claim 1, wherein the method further comprises sequencing genomic DNA isolated from nucleated blood cells from the patient and measuring the variant sequences that are not derived from the nucleated blood cells.

10. The method of claim 1, wherein prior to step (a), the method comprises sequencing tumor DNA from the patient and identifying a plurality of sequence variations in the tumor, and wherein the amount of ctDNA in step (a) by monitored by assaying the identified sequence variations.
